(19) United States
(12) Patent Application Publication
(10) Pub. No.: US 2024/0023724 A1
(43) Pub. Date: Jan. 25, 2024

(54) MEDICAL IMAGE DIAGNOSIS APPARATUS

(71) Applicant: CANON MEDICAL SYSTEMS CORPORATION, Otawara-shi (JP)

(72) Inventor: Yugo TABATA, Nasushiobara (JP)

(73) Assignee: CANON MEDICAL SYSTEMS CORPORATION, Otawara-shi (JP)

(21) Appl. No.: 18/351,582

(22) Filed: Jul. 13, 2023

(30) Foreign Application Priority Data
Jul. 19, 2022 (JP) 2022-115109

(51) Int. Cl. A47C 27/08; A61B 5/055

(52) U.S. Cl. CPC A47C 27/081; A61B 5/055

(57) ABSTRACT

A medical image diagnosis apparatus according to one or more embodiments includes an air mattress, an air supplier, and processing circuitry. The air mattress is configured to be placed on a bed. The air mattress includes a plurality of air cells partitioning an inside of the air mattress into a plurality of regions to seal air supplied to the air cells. The air supplier supplies air to the air cells of the air mattress. The processing circuitry is configured to control the supply of air to the air cells of the air mattress so that a top surface of the air mattress may partially protrude upward from a flat state of the air mattress.

### CROSS-REFERENCE TO RELATED APPLICATION

This application is based upon and claims the benefit of priority from the prior Japanese Patent Application No. 2022-115109, filed on Jul. 19, 2022, the entire contents of which are incorporated herein by reference.

### FIELD

Embodiments described herein and illustrated in the accompanying drawings relate to a medical image diagnosis apparatus.

### BACKGROUND

Conventionally, magnetic resonance imaging (MRI) apparatuses were known in the medical field. Such apparatuses excite nuclear spins of a subject placed in a static magnetic field by means of a radio frequency (RF) signal at the Larmor frequency and regenerate a magnetic resonance (MR) signal that is generated at the subject as the excitation occurs to generate a medical image.

When taking an image of a subject using such the magnetic resonance imaging apparatus, the operator may put a mattress between a bed and a portion of the subject (e.g., a knee or the head) so that the subject on the bed may be in a comfortable position.

In order to place the mattress between the bed and the portion of the subject, however, the operator may need to lift the portion of the subject. In such a case, the physical load of the operator may be increased, and the time needed for the preparation of the imaging may be elongated.

### BRIEF DESCRIPTION OF THE DRAWINGS

FIG. 1 is a block diagram illustrating an example of a configuration of a magnetic resonance imaging apparatus according to a first embodiment.

FIG. 2 is a plan view of an example of a configuration of an air mattress in the magnetic resonance imaging apparatus according to the first embodiment.

FIG. 3 is a cross-sectional view of the air mattress of the magnetic resonance imaging apparatus according to the first embodiment, when the air mattress is not supplied with air.

FIG. 4 is a cross-sectional view of the air mattress of the magnetic resonance imaging apparatus according to the first embodiment, when the air mattress is supplied with air.

FIG. 5 is a flowchart showing an example of an operation of the magnetic resonance imaging apparatus according to the first embodiment.

FIG. 6 is an explanatory diagram for explaining a step of detecting a supported portion and a step of determining air cells to be supplied with air in the example of the operation of the magnetic resonance imaging apparatus according to the first embodiment.

FIG. 7 is an explanatory diagram for explaining a step of supplying air to the air cells to be supplied with air in the example of the operation of the magnetic resonance imaging apparatus according to the first embodiment.

FIG. 8 is a flowchart showing an example of an operation of a magnetic resonance imaging apparatus according to a second embodiment.

FIG. 9 is an explanatory diagram for explaining a step of supplying air to air cells to be supplied with air in an example of an operation of the magnetic resonance imaging apparatus according to the second embodiment.

FIG. 10 is a flowchart showing an example of an operation of a magnetic resonance imaging apparatus according to a third embodiment.

FIG. 11 is a block diagram showing an example of a configuration of a magnetic resonance imaging apparatus according to a fourth embodiment.

FIG. 12 is a flowchart showing an example of an operation of the magnetic resonance imaging apparatus according to the fourth embodiment.

FIG. 13 is an explanatory diagram for explaining a step of acquiring a side view image of a subject in the example of the operation of the magnetic resonance imaging apparatus according to the fourth embodiment.

FIG. 14 is an explanatory diagram for explaining a step of detecting the degree of curve on a back side of the subject in the magnetic resonance imaging apparatus according to the fourth embodiment.

FIG. 15 is an explanatory diagram for explaining a step of supplying air to air cells to be supplied with air in the example of the operation of the magnetic resonance imaging apparatus according to the fourth embodiment.

FIG. 16 is a block diagram showing an example of a configuration of a magnetic resonance imaging apparatus according to a fifth embodiment.

FIG. 17 is a flowchart showing an example of an operation of the magnetic resonance imaging apparatus according to the fifth embodiment.

FIG. 18 is an explanatory diagram for explaining a step of extracting subject-specific information in the example of the operation of the magnetic resonance imaging apparatus according to the fifth embodiment.

FIG. 19 is an explanatory diagram for explaining a step of determining air cells to be supplied with air and an amount of air supplied to the air cells to be supplied with air in the example of the operation of the magnetic resonance imaging apparatus according to the fifth embodiment.

FIG. 20 is an explanatory diagram for explaining the step of determining the amount of air to be supplied in the example of the operation in the magnetic resonance imaging apparatus according to the fifth embodiment.

### DETAILED DESCRIPTION

Embodiments of the medical image diagnosis apparatus will now be described with reference to the accompanying drawings. Although the following descriptions relate to embodiments of a magnetic resonance imaging apparatus, which is an example of the medical image diagnosis apparatus, the medical image diagnosis apparatus is not limited to the magnetic resonance imaging apparatus. The medical image diagnosis apparatus may be a medical image apparatus with a bed. For example, the medical image diagnosis apparatus may be applied to an X-ray computed tomography apparatus. In the following descriptions, elements having substantially the same function and configuration have the same reference numerals. Explanation for such an element is repeated only when it is necessary to do so.

### First Embodiment

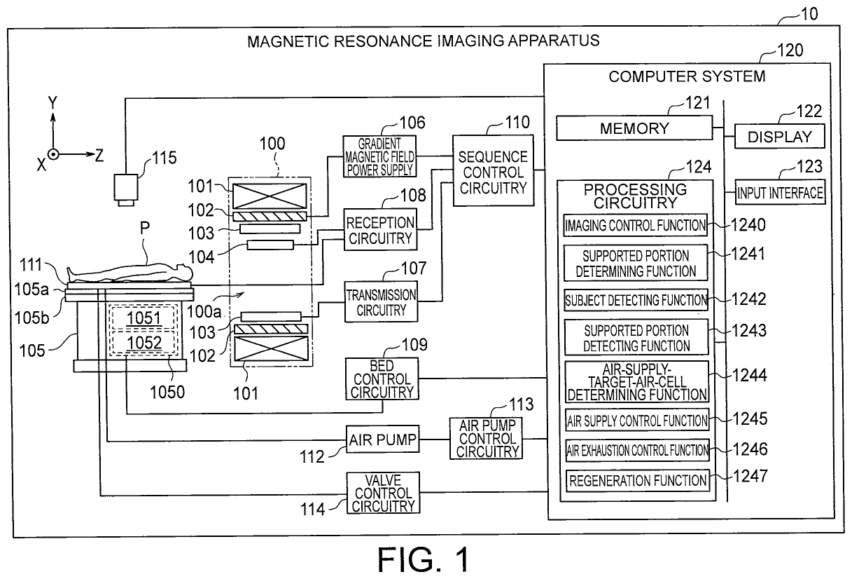

FIG. 1 is a block diagram showing an example of a configuration of a magnetic resonance imaging apparatus 10 according to a first embodiment. As shown in FIG. 1, the magnetic resonance imaging apparatus 10 according to the first embodiment includes a gantry 100 and a bed 105. The gantry 100 includes a static magnetic field magnet 101, a gradient magnetic field coil 102, a transmission coil 103, and a reception coil 104. The bed 105 includes a top panel 105a, a top panel support 105b, and a top panel driver 1050. The magnetic resonance imaging apparatus 10 also includes a gradient magnetic field power supply 106, transmission circuitry 107, reception circuitry 108, bed control circuitry 109, and sequence control circuitry 110. The magnetic resonance imaging apparatus 10 further includes an air mattress 111, an air pump 112, an air pump control circuitry 113, a valve control circuitry 114, a ceiling camera 115, and a computer system 120. The air pump 112, the air pump control circuitry 113, and the valve control circuitry 114 are examples of an air supplier and an air exhauster. The ceiling camera 115 is an example of an imaging device.

The magnetic resonance imaging apparatus 10 does not include a subject P. The configuration of the magnetic resonance imaging apparatus 10 is not limited to that shown in FIG. 1. In other words, the configuration of the magnetic resonance imaging apparatus 10 may be arbitrarily determined. For example, the elements in the sequence control circuitry 110 and the computer system 120 may be arbitrarily combined or separated.

The gantry 100 is a device configured to take an image of the subject P using a magnetic field. The gantry 100 has a bore 100a having a cross section in a substantially circular shape (including an elliptical shape), the cross section being orthogonal to a central axis of the bore 100a. The bore 100a corresponds to an imaging space in which the subject P is placed when an image is taken. The static magnetic field magnet 101, the gradient magnetic field coil 102, the transmission coil 103, and the reception coil 104 are housed in the gantry 100 around the bore 100a. The reception coil 104 is located on an inner side relative to the other coils 102 and 103 around the bore 100a. The transmission coil 103 is located on the outer side of the reception coil 104. The gradient magnetic field coil 102 is located on the outer side of the transmission coil 103. The static magnetic field magnet 101 is located on the outer side of the gradient magnetic field coil 102.

The static magnetic field magnet 101 is a magnet in a substantially cylindrical shape having a hollow. The static magnetic field magnet 101 generates a static magnetic field in the imaging space. The static magnetic field magnet 101 may be a superconducting magnet. The superconducting magnet includes, for example, a container filled with a cooling agent such as liquid helium and a superconducting coil immersed in the container. If the static magnetic field magnet 101 is a superconducting magnet, the static magnetic field magnet 101 is supplied with an electric current (power supply) from a static magnetic field power supply to excite. As another example, the static magnetic field magnet 101 may be a permanent magnet. In such a case, the magnetic resonance imaging apparatus 10 may not include a static magnetic field power supply. The static magnetic field power supply may be disposed independently of the magnetic resonance imaging apparatus 10.

The gradient magnetic field coil 102 is a coil formed in a shape of a hollow cylinder. The gradient magnetic field coil 102 is disposed within the static magnetic field magnet 101, and has an X coil, a Y coil, and a Z coil corresponding to an X axis, a Y axis, and a Z axis which are orthogonal to each other. A “Z axis direction” is a direction along a magnetic flux of a static magnetic field generated by the static magnetic field magnet 101. A “Y axis direction” is a vertical direction and an “X axis direction” is perpendicular to the Z axis and the Y axis. The gradient magnetic field coil 102 generates a gradient magnetic field to be superimposed on the static magnetic field in the imaging space. Specifically, the X coil, the Y coil, and the Z coil included in the gradient magnetic field coil 102 are separately supplied with a current (power supply) from the gradient magnetic field power supply 106 and generate a gradient magnetic field, the magnetic field strength of which linearly changes along each of the X, Y, and Z axes.

The gradient magnetic field power supply 106 supplies the current to the gradient magnetic field coil 102 under the control of the sequence control circuitry 110 to generate the gradient magnetic field in the imaging space. Specifically, the gradient magnetic field power supply 106 separately supplies a current to each of the X coil, the Y coil, and the Z coil of the gradient magnetic field coil 102 to generate the gradient magnetic field in the imaging space, where the gradient magnetic field linearly changes in a readout direction, a phase encoding direction, and a slice direction, which are orthogonal to each other. In the following descriptions, the gradient magnetic field in the readout direction is called “readout gradient magnetic field,” the gradient magnetic field in the phase encoding direction is called “phase encoding gradient magnetic field,” and the gradient magnetic field in the slice direction is called “slice gradient magnetic field.”

The readout gradient magnetic field, the phase encoding gradient magnetic field, and the slice gradient magnetic field are superimposed on the static magnetic field generated by the static magnetic field magnet 101. The readout gradient magnetic field, the phase encoding gradient magnetic field, and the slice gradient magnetic field superimposed on the static magnetic field provides spatial position information to a magnetic resonance signal generated from the subject P. Specifically, the readout gradient magnetic field changes the frequency of the magnetic resonance signal in accordance with a location in the readout direction to provide position information in the readout direction to the magnetic resonance signal. The phase encoding gradient magnetic field changes the phase of the magnetic resonance signal in the phase encoding direction to provide position information in the phase encoding direction to the magnetic resonance signal. The slice gradient magnetic field provides position information in the slice direction to the magnetic resonance signal. For example, if the imaging region is a slice region (2D imaging), the slice gradient magnetic field is used to determine the direction, the thickness, and the number of the slice region. If the imaging region is a volume region (3D imaging), the slice gradient magnetic field is used to change the phase of the magnetic resonance signal in accordance with the position in the slice direction. Thus, the axis in the readout direction, the axis in the phase encoding direction, and the axis in the slice direction form a logical coordinate system for defining the slice region or volume region to be imaged.

The transmission coil 103 is disposed on the inner side of the gradient magnetic field coil 102. The transmission coil 103 is in a substantially cylindrical shape having a hollow, and applies a high frequency magnetic field to the subject P disposed in the imaging space. Specifically, the transmission coil 103 applies the high frequency magnetic field to the subject P in the imaging space within the transmission coil 103 in response to an RF (Radio Frequency) pulse signal supplied from the transmission circuitry 107.

The transmission circuitry 107 supplies the RF pulse signal to the transmission coil 103 under the control of the sequence control circuitry 110. Specifically, the transmission circuitry 107 outputs to the transmission coil 103 the RF pulse signal that corresponds to the Larmor frequency specific to the target atomic nucleus in the static magnetic field. For example, the transmission circuitry 107 includes a pulse generator, an RF generator, a modulator, and an amplifier. The pulse generator generates a waveform of the RF pulse signal. The RF generator generates an RF signal at a resonance frequency. The modulator modulates the amplitude of the RF signal generated by the RF generator using the waveform generated by the pulse generator to form the RF pulse signal. The amplifier amplifies the RF pulse signal generated by the modulator and outputs the amplified RF pulse signal to the transmission coil 103. The transmission circuitry 107 may be included in the gantry 100.

The reception coil 104 is disposed on the inner side of the transmission coil 103. The reception coil 104 receives the magnetic resonance signal generated from the subject P due to the influence of the high frequency magnetic field, and outputs the received magnetic resonance signal to the reception circuitry 108. A single coil may be used as both the reception coil 104 and the transmission coil 103.

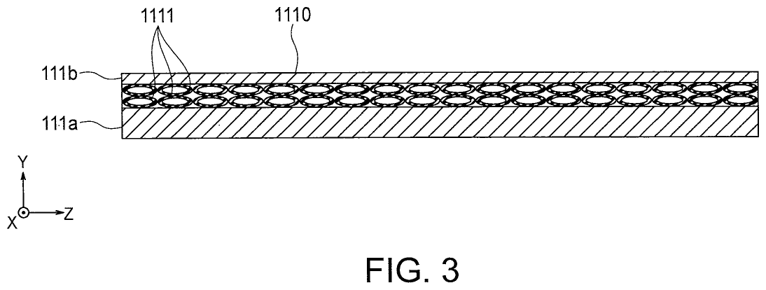

The reception circuitry 108 receives magnetic resonance signals outputted from the reception coil 104 and a reception coil 111b of an air mattress 111 that will be described later (see FIG. 3). The reception circuitry 108 generates magnetic resonance data based on the received magnetic resonance signal, and outputs the generated magnetic resonance data to the sequence control circuitry 110. For example, the reception circuitry 108 includes a selector, a pre-stage amplifier, a phase detector, and an analog-to-digital (A/D) converter. The selector selectively receives one of the magnetic resonance signals outputted from the reception coil 104 and the reception coil 111b of the air mattress 111. The pre-stage amplifier amplifies the power of the magnetic resonance signal outputted from the selector. The phase detector detects the phase of the magnetic resonance signal outputted from the pre-stage amplifier. The A/D converter converts an analog signal outputted from the phase detector to a digital signal to generate the magnetic resonance data, and outputs the generated magnetic resonance data to the sequence control circuitry 110. The above-described operations explained as being performed by the reception circuitry 108 are not necessarily performed by the reception circuitry 108, and some of the operations (e.g., the operations performed by the A/D converter) may be performed by reception coil 104 or the reception coil 111b of the air mattress 111. The reception circuitry 108 may be included in the gantry 100.

The bed 105 is a device on which the subject P is placed for being moved into the imaging space of the gantry 100. The bed 105 includes the top panel 105a on which the subject P is placed, the top panel support 105b configured to support the top panel 105a, and the top panel driver 1050 configured to drive the top panel 105a. The top panel support 105b is disposed under the top panel 105a and supports the top panel 105a so that the top panel driver 1050 may drive the top panel 105a. The bed 105 is configured so that the longitudinal direction of the top panel 105a is parallel to a central axis (extending in the Z axis direction) of the static magnetic field magnet 101. The top panel driver 1050 drives the top panel 105a to move up or down along the Y axis direction, and to move in the longitudinal direction of the top panel 105a along the Z axis direction. Specifically, the top panel driver 1050 includes a vertical driving unit 1051 configured to move the top panel 105a up and down, and a horizontal driving unit 1052 configured to move the top panel 105a in the horizontal direction of the top panel 105a.

When an image of the subject P is taken at the gantry 100, the vertical driving unit 1051 moves up the top panel 105a together with the top panel support 105b to a lift position corresponding to the height of the imaging space of the gantry 100. The horizontal driving unit 1052 horizontally moves the top panel 105a, which has been lifted to the lift position by the vertical driving unit 1051, in the longitudinal direction of the top panel 105a toward the imaging space of the gantry 100. The top panel support 105b is not moved to the imaging space but remains at the lift position. After an image of subject P is taken at the gantry 100, the horizontal driving unit 1052 returns the top panel 105a from the imaging space of the gantry 100 to the lift position. The vertical driving unit 1051 then lowers the top panel 105a, which has been returned to the lift position by the horizontal driving unit 1052, together with the top panel support 105b.

The vertical driving unit 1051 includes a first driving source that may generate a power to move the top panel 105a up and down, and a first driving force conveying member that may convey the driving force of the first driving source to the top panel 105a. For example, the first driving source may include a hydraulic cylinder and a hydraulic pressure control unit. The hydraulic cylinder compresses and extends according to the oil pressure. The hydraulic pressure control unit controls the compression and the extension of the hydraulic cylinder under the control of the bed control circuitry 109. The hydraulic pressure control unit may include, for example, a hydraulic pump configured to send pressurized oil to the hydraulic cylinder under the control of the bed control circuitry 109. The first driving force conveying member may include an arm of which a lower end is rotatably supported by a bottom part of the bed 105 and an upper end is fixed to a lower surface of the top panel support 105b. The arm may be connected to the hydraulic cylinder and rotate in the vertical direction as the hydraulic cylinder compresses or extends. The horizontal driving unit 1052 may include, for example, a second driving source that may generate a power to move the top panel 105a in the longitudinal direction of the top panel 105a, and a second driving force conveying member that may convey the driving force of the second driving source to the top panel 105a. The second driving source may be, for example, a motor. The second driving force conveying member may include a pulley that rotates as the motor rotates, and a belt that is disposed at the periphery of the pulley to convert the rotational motion of the motor to a translational movement of the top panel 105a. The pulley and the belt may be provided to each of the top panel support 105b and the top panel 105a.

Under the control of the computer system 120, the bed control circuitry 109 controls the top panel driver 1050 to drive the top panel 105a. For example, the bed control circuitry 109 controls the power supply to the top panel driver 1050 to thereby control the driving of the top panel 105. For example, the bed control circuitry 109 receives a user's instruction to move the top panel 105a in the vertical direction or the longitudinal direction of the top panel 105a via the computer system 120. The bed control circuitry 109 then performs a control operation to drive the top panel 105a in accordance with the received instruction.

The sequence control circuitry 110 takes an image of the subject P by driving the gradient magnetic field power supply 106, the transmission circuitry 107, and the reception circuitry 108 in accordance with sequence information sent from the computer system 120. The sequence information defines the procedure of the imaging. For example, the sequence information may define the strength of a current supplied by the gradient magnetic field power supply 106 to the gradient magnetic field coil 102, and the timing at which the gradient magnetic field power supply 106 supplies the current to the gradient magnetic field coil 102. The sequence information may define the strength of the RF pulse signal supplied from the transmission circuitry 107 to the transmission coil 103, and the timing at which the RF pulse signal is applied. The sequence information may also define the timing at which the reception circuitry 108 detects the magnetic resonance signal. More specifically, the sequence control circuitry 110 receives the magnetic resonance data outputted from the reception circuitry 108 as a result of the imaging based on the sequence information, and causes a memory 121 to store the received magnetic resonance data. At this time, position information relating to the readout direction, the phase-out direction, and the slice direction is added to the magnetic resonance data stored in the memory 121 by the readout gradient magnetic field, the phase encoding gradient magnetic field, and the slice gradient magnetic field described above. As a result, the magnetic resonance data is stored in the memory 121 as k-space data representing a two-dimensional or three-dimensional k-space. The sequence control circuitry 110 may be a processor.

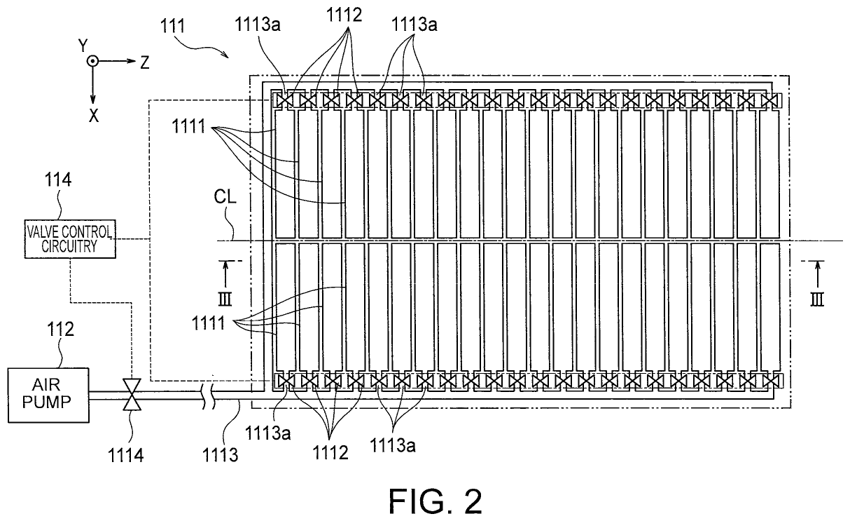

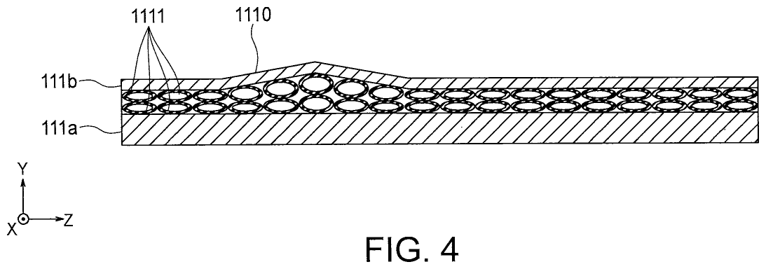

FIG. 2 is a plan view of an example of a configuration of the air mattress 111 included in the magnetic resonance imaging apparatus 10 according to the first embodiment. FIG. 3 is a cross-sectional view of the air mattress 111 in the magnetic resonance imaging apparatus 10 according to the first embodiment, when the air mattress is not supplied with air. FIG. 4 is a cross-sectional view of the air mattress 111 of the magnetic resonance imaging apparatus 10 according to the first embodiment, when the air mattress 111 is supplied with air. FIGS. 3 and 4 are cross sectional views taken along line III-III in FIG. 2. In FIGS. 3 and 4, the configuration of the air mattress 111 is simplified except for air cells 1111. The air mattress 111 is able to support the subject P on the bed 105 using air pressure. The air mattress 111 is placed on the top panel 105a of the bed 105. As shown in FIG. 2, in the plan view, the air mattress 111 has a substantially rectangular shape with a long side extending in the longitudinal direction of the top panel 105a (i.e., the Z axis direction). In FIG. 2, the outer edge of the air mattress 111 is indicated by a dash and double-dot line forming a rectangle surrounding all of the air cells 1111. As shown in FIGS. 3 and 4, the air mattress 111 has a structure in which a cushion 111a, a plurality of air cells 1111, and a reception coil 111b are layered. Furthermore, as shown in FIG. 2, the air mattress 111 includes a plurality of valves 1112, a vent tube 1113, and an exhaust valve 1114 that is a part of the air exhauster.

As shown in FIG. 3, the cushion 111a is a bottom part (bottommost layer) of the air mattress 111. The cushion 111a may be formed of a resin material such as urethane or a polyester so as to have flexibility.

The air cells 1111 are disposed above the cushion 111a. The air cells 1111 are bag-like members used for partition an inside of the air mattress 111 into a plurality of regions. In the example shown in FIG. 2, each of the air cells 1111 has the long side extending in the width direction of the air mattress 111, the width direction being orthogonal to the longitudinal direction and the vertical direction of the top panel 105a. Thus, the air cells 1111 each have a long side extending in the X axis direction. The air cells 1111 may be separately supplied with air, may be separately sealed, and may separately exhaust the supplied air. When supplied with air, the air cells 1111 expand due to air pressure. As the air cells 1111 are expanding, portions of a top surface 1110 of the air mattress 111 corresponding to the expanding air cells 1111 may protrude upward. The word “upward” here means the positive direction on the Y axis (the direction of the arrow on the Y axis) shown in FIG. 1. A “downward” direction means a direction opposite to the “upward” direction, which is the negative direction on the Y axis. The material of the air cells 1111 is not limited as long as the air cells 1111 are flexible enough to be deformed by air pressure, and sufficiently sealed so as not to leak supplied air. For example, the air cells 1111 may be formed of a resin material.

As shown in FIGS. 2 to 4, the air cells 1111 are arranged in the horizontal direction (i.e., the X axis direction and the Z axis direction) and the vertical direction (i.e., the Y axis direction). This ensures the degree of freedom of the location and the amount of the protrusion of the top surface 1110 of the air mattress 111. The air cells 1111 may be arranged to cover substantially the entire area of the cushion 111a. This enables the top surface 1110 of the air mattress 111 to protrude to support any desired portion of the subject P regardless of whether the head portion of the subject P is on the gantry 100 side or the opposite side. As shown in FIG. 2, in the plan view, the air cells 1111 are axis-symmetrically arranged relative to a center line CL of the air mattress 111, which is parallel to the longitudinal direction of the top panel 105a. Specifically, two lines of the air cells 1111 are arranged in the X axis direction on both sides of the center line CL. The number of air cells 1111 in each of the two lines arranged in the Z axis direction (i.e., the longitudinal direction of the top panel 105a) is the same. By axis-symmetrically arranging the air cells 1111 relative to the center line CL of the air mattress 111, the top surface 1110 of the air mattress 111 may be caused to protrude to support either the left or the right leg of the subject R Furthermore, as shown in FIG. 3, the air cells 1111 are vertically arranged in two layers.

The air pump 112 is connected to the air cells 1111 via the vent tube 1113. The air pump 112 may be able to supply air to the air cells 1111. In the example of FIG. 2, the air pump 112 is connected to an end of the vent tube 1113. The other end side of the vent tube 1113 is branched into a plurality of branch portions 1113a each corresponding to one of the air cells 1111. The branch portions 1113a are connected to the corresponding air cells 1111. Specifically, each of the air cells 1111 is connected to a corresponding branch portions 1113a at an outer end in the longitudinal direction thereof (i.e., the X axis direction). A valve 1112 is provided to each of the branch portions 1113a in order to allow the supply and the discharge of air separately to and from each air cell 1111. The exhaust valve 1114 that is able to open the vent tube 1113 to expose the inside thereof to the outside air is disposed on the vent tube 1113 between the air pump 112 and the branch portions 1113a. The valves 1112 and the exhaust valve 1114 may be separately opened or closed by electrical control. The opening degree of the valves 1112 and the exhaust valve 1114 may be changeable by electrical control so as to adjust the amount of supply and discharge of air to and from the air cells 1111.

The air pump 112 is able to supply air to the air cells 1111 via the vent tube 1113 when the valves 1112 are open and the exhaust valve 1114 is closed. If the air cells 1111 are supplied with air, the air cells 1111 may expand as shown in FIG. 4. The expansion of the air cells 1111 causes the top surface 1110 of the air mattress 111 to protrude upward at a portion corresponding to the expanding air cells 1111. The closing of the valves 1112 corresponding to the air cells 1111 supplied with air makes it possible to seal the air cells 1111 to maintain the expanded state of the air cells 1111. The opening of the valves 1112 corresponding to the air cells 1111 supplied with air and sealed and the opening of the exhaust valve 1114 make it possible to exhaust air from the air cells 1111.

The arrangement of the valves 1112, the vent tube 1113, and the air pump 112 is not limited to that shown in FIG. 2 as long as air may be supplied to and exhausted from the air cells 1111. For example, the air pump 112 may include a supply pump (i.e., compressor) and a discharge pump (i.e., inspiratory pump). In this case, the vent tube 1113 may include a supply tube connecting the supply pump and the air cells 1111, and a discharge tube connecting the discharge pump and the air cells 1111. Each of the valves 1112 may have a supply valve disposed on the supply tube and a discharge valve disposed on the discharge tube. In this case, the air supply and the air discharge of the air cells 1111 may be selectively switched by selectively driving the supply pump or the discharge pump, and selectively opening the supply valve or the discharge valve.

The reception coil 111b is disposed above the air cells 1111, and forms an upper end portion (i.e., uppermost layer) of the air mattress 111. The reception coil 111b receives the magnetic resonance signal generated from the subject P when an image of the subject P is taken. Specifically, when an image of the subject P is taken, the reception coil 111b is disposed under the back of the subject P as a part of the air mattress 111. The reception coil 111b then receives the magnetic resonance signal generated from the subject P due to the influence of the high frequency magnetic field applied by the transmission coil 103. The reception coil 111b outputs the received magnetic resonance signal to the reception circuitry 108. For example, the reception coil 111b may be a surface coil, or a phased array coil including a combination of a plurality of surface coils as a coil element.

The air pump control circuitry 113 controls the driving of the air pump 112 under the control of the computer system 120. For example, the air pump control circuitry 113 controls the power supply to the air pump 112 to control the driving of the air pump 112. The air pump control circuitry 113 control the power supply to the air pump 112 to control the supply of air from the air pump 112 to the air cells 1111. For example, the air pump control circuitry 113 has power supply circuitry configured to supply power to the air pump 112 and power supply control circuitry configured to control the power supply circuitry. The power supply control circuitry may be, for example, a processor. At least a part of the air pump control circuitry 113 may be included in the computer system 120.

The valve control circuitry 114 electrically controls the opening and the closing of the valves 1112 and the exhaust valve 1114 under the control of the computer system 120. For example, the valve control circuitry 114 separately controls the opening and the closing of each of the valves 1112 and the exhaust valve 1114 by outputting an electrical signal instructing the opening or closing of each of the valves 1112 and the exhaust valve 1114 to the corresponding one of the valves 1112 and the exhaust valve 1114. The valve control circuitry 114 may control the supply or discharge amount of air to or from the air cells 1111 by separately outputting an electrical signal used for controlling the degree of opening of the valves 1112 and the exhaust valve 1114 to each of the valves 1112 and the exhaust valve 1114. For example, the valve control circuitry 114 is a processor. The valve control circuitry 114 may be included in the computer system 120.

The ceiling camera 115 is a camera fixed to the ceiling of a room (e.g., shielded room) in which the gantry 100 and the bed 105 are disposed. The ceiling camera 115 may include a solid-state imager such as a complementary metal oxide semiconductor (CMOS) sensor. The ceiling camera 115 takes an image of the subject P on the air mattress 111 using visible light, under the control of the computer system 120. The ceiling camera 115 may be disposed immediately above the air mattress 111 so that the air mattress 111 is on an optical axis of the ceiling camera 115, for example. The arrangement of the ceiling camera 115 is not limited to the above example, but the ceiling camera 115 may be disposed to a position that is horizontally shifted from the position immediately above the air mattress 111 as long as the top surface of the air mattress 111 is within the viewing angle of the ceiling camera 115. The ceiling camera 115 may be arranged so that the optical axis may be tilted relative to a surface normal direction (Y axis direction) of the air mattress 111.

The computer system 120 is a system configured to control the entire operation of the magnetic resonance imaging apparatus 10. For example, the computer system 120 controls the generation of a magnetic resonance image, and the supply and the discharge of air to and from the air cells 1111. As shown in FIG. 1, the computer system 120 includes the memory 121, a display 122, an input interface 123, and processing circuitry 124.

The memory 121 is a non-transitory storage device configured to store various kinds of information. Examples of the memory 121 include a hard disk drive (HDD), a solid state drive (SSD), and an integrated circuit storage device. The memory 121 may store, for example, a control program for controlling the magnetic resonance imaging apparatus 10 and various kinds of data used for executing the control program. Instead of the HDD or the SSD, the memory 121 may be a driving device configured to read and write various kinds of information between such portable storage media as a compact disc (CD), a digital versatile disc (DVD), and a flash memory, or a semiconductor memory element such as a random access memory (RAM).

The display 122 displays various kinds of information. For example, the display 122 outputs a graphical user interface (GUI) used for receiving the medical image generated by the processing circuitry 124 and various instructions from the operator. For example, a liquid crystal display (LCD), a cathode ray tube (CRT) display, an organic electro luminescence display (OELD), a plasma display, or any other suitable display may be arbitrarily used as the display 122. The display 122 may be included in the gantry 100. The display 122 may be of a desktop type, or a tablet configured to wirelessly communicate with the processing circuitry 124.

The input interface 123 receives various inputs from the operator. The input interface 123 converts a received input to an electrical signal and outputs the electrical signal to the processing circuitry 124. For example, the input interface 123 receives an imaging condition from the operator. The imaging condition may include order information instructing a portion of the subject P to be imaged by the gantry 100. The way of inputting the order information is not limited to the above-described manner, but the order information may be inputted independently of the imaging information. The input interface 123 may be selected from, for example, one or more of a mouse device, a keyboard, a track ball, a switch, a button, a joystick, a touchpad, and a touch panel display.

The processing circuitry 124 controls the entire operations of the magnetic resonance imaging apparatus 10 in response to the electrical signal outputted from the input interface 123, the electrical signal corresponding to an inputted instruction. For example, the processing circuitry 124 has an imaging control function 1240, a supported portion determining function 1241, a subject detecting function 1242, a supported portion detecting function 1243, an air-supply-target-air-cell determining function 1244, an air supply control function 1245, an air exhaustion control function 1246, a regeneration function 1247.

For example, the respective processing functions of the imaging control function 1240, the supported portion determining function 1241, the subject detecting function 1242, the supported portion detecting function 1243, the air-supply-target-air-cell determining function 1244, the air supply control function 1245, the air exhaustion control function 1246, and the regeneration function 1247, which are elements of the processing circuitry 124 shown in FIG. 1, are stored in the memory 121 in the form of programs executable by computers. The processing circuitry 124 is, for example, a processor. The processor corresponding to the processing circuitry 124 performs a function corresponding to a program by reading the program from the memory 121 and executing the program. In other words, after reading a program, the processing circuitry 124 has a corresponding function shown in the processing circuitry 124 in FIG. 1.

Although FIG. 1 indicates that the processing functions such as the imaging control function 1240, the supported portion determining function 1241, the subject detecting function 1242, the supported portion detecting function 1243, the air-supply-target-air-cell determining function 1244, the air supply control function 1245, the air exhaustion control function 1246, and the regeneration function 1247 are performed by a single processor corresponding to the processing circuitry 124, the embodiment is not limited to such a configuration. For example, the processing circuitry 124 may include a plurality of independent processors, each executing a program to perform a corresponding processing function. Furthermore, the processing functions of the processing circuitry 124 may be arbitrarily assigned to separate processing circuits or a single processing circuit.

The imaging control function 1240 generates sequence information based on an imaging condition indicated by the electrical signal outputted from the input interface 123. The imaging control function 1240 outputs the generated sequence information to the sequence control circuitry 110.

The supported portion determining function 1241 determines a supported portion of the subject P, which will be supported by a protruding portion of the top surface 1110 of the air mattress 111, based on the order information acquired by the input action of the operator. The order information is an example of input information. The supported portion may be, for example, a knee, the back, or the neck.

The subject detecting function 1242 detects the subject P on the air mattress 111 based on the image taken by the ceiling camera 115.

The supported portion detecting function 1243 detects the supported portion, which is determined by the supported portion determining function 1241, of the subject P detected by the subject detecting function 1242.

The air-supply-target-air-cell determining function 1244 determines air cells, which will be supplied with air (“air cells to be supplied with air”) among the air cells 1111 included in the air mattress 111 based on the image taken by the ceiling camera 115. Specifically, the air-supply-target-air-cell determining function 1244 determines the air cells to be supplied with air based on the image taken by the ceiling camera 115 and the supported portion determined by the supported portion determining function 1241. More specifically, the air-supply-target-air-cell determining function 1244 determines the air cells to be supplied with air based on the supported portion, which is detected by the supported portion detecting function 1243, of the subject P detected in the image taken by the subject detecting function 1242.

The air supply control function 1245 controls the supply of air to the air cells 1111 of the air mattress 111, the air cells 1111 being flat in the initial state and locally protruded upward from the top surface 1110 of the air mattress 111. Specifically, the air supply control function 1245 controls the supply of air by the air pump 112 so that only the air cells to be supplied with air determined by air-supply-target-air-cell determining function 1244 are supplied with air. More specifically, the air supply control function 1245 outputs a control signal to the air pump control circuitry 113 for controlling the air pump control circuitry 113, and a control signal to the valve control circuitry 114 for controlling the valve control circuitry 114, thereby controlling the supply of air to the air cells to be supplied with air.

The air exhaustion control function 1246 controls the discharge of air from the air cells 1111. Specifically, the air exhaustion control function 1246 outputs the control signal for controlling the valve control circuitry 114 to the valve control circuitry 114 to control the discharge of air from the air cells to be supplied with air.

The regeneration function 1247 regenerates a medical image from the magnetic resonance data (k-space data) received by the sequence control circuitry 110. Specifically, the regeneration function 1247 reads the magnetic resonance data received by the sequence control circuitry 110 from the memory 121. The sequence control circuitry 110 then performs regeneration processing such as Fourier transform on the read magnetic resonance data to generate a two-dimensional or three-dimensional medical image. The regeneration function 1247 causes the memory 121 to store the generated medical image.

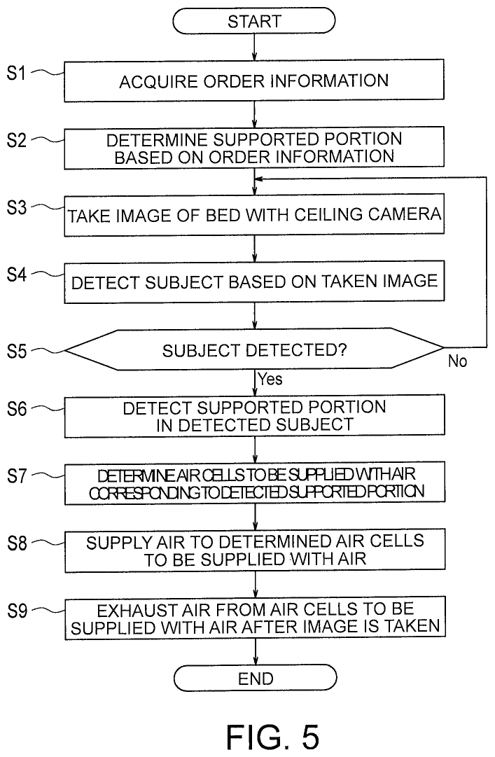

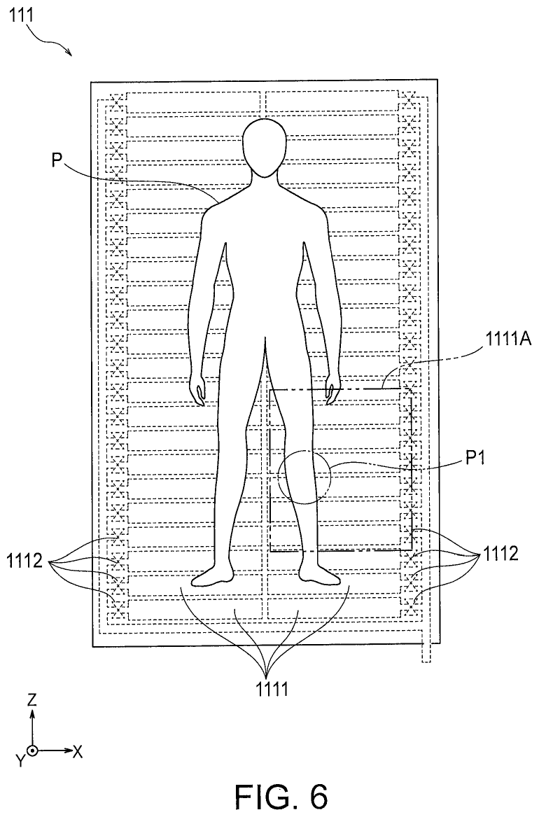

An example of an operation of the magnetic resonance imaging apparatus according to the first embodiment 10 having the above-described configuration will now be described. FIG. 5 is a flowchart showing an example of an operation of the magnetic resonance imaging apparatus 10 according to the first embodiment. FIG. 6 is an explanatory diagram for explaining a step of detecting a supported portion and a step of determining air cells to be supplied with air in the example of the operation of the magnetic resonance imaging apparatus 10 according to the first embodiment. In the initial state in FIG. 5, no air has been supplied to the air cells 1111, and all the valves 1112, 1114 are closed. Furthermore, in the initial state in FIG. 5, a subject P lies on the back on the air mattress 111, as shown in FIG. 6.

From the initial state, the supported portion determining function 1241 acquires order information inputted to the processing circuitry 124, as shown in FIG. 5 (step 51). The order information may be included in the imaging condition outputted from the input interface 123 to the processing circuitry 124. Alternatively, the order information may be included in sequence information generated by the sequence control circuitry 110 based on the imaging condition, and inputted by the sequence control circuitry 110 to the processing circuitry 124. Alternatively, the order information may be inputted to the processing circuitry 124 as information designated by the operator separately from the imaging condition and the sequence information.

After the order information is acquired, the supported portion determining function 1241 determines a supported portion based on the acquired order information (step S2). The supported portion determining function 1241 causes the memory 121 to store supported portion data indicating the determined supported portion.

After the supported portion is determined, the air supply control function 1245 controls the ceiling camera 115 to take an image of the bed 105 on which the air mattress 111 is placed (step S3). The air supply control function 1245 causes the memory 121 to store the image taken by the ceiling camera 115.

After the image is taken by the ceiling camera 115, the subject detecting function 1242 detects the subject P based on the image (step S4). Specifically, the subject detecting function 1242 reads the image taken by the ceiling camera 115 from the memory 121, and detects the subject P by performing an image recognition operation such as pattern matching and image segmentation on the read image. The subject detecting function 1242 causes the memory 121 to store subject detection data indicating the detected subject P.

Thereafter, the supported portion detecting function 1243 determines whether the subject detecting function 1242 detects the subject P (step S5). For example, the supported portion detecting function 1243 determines that the subject P is detected if the memory 121 stores the subject detection data.

If the subject P is detected (step S5: Yes), the supported portion detecting function 1243 detects the supported portion from the detected subject P (step S6). Specifically, the supported portion detecting function 1243 reads the supported portion data and the subject detection data from the memory 121. The supported portion detecting function 1243 then performs the image recognition operation such as pattern matching and image segmentation on the read subject detection data to detect a supported portion corresponding to the read supported portion data. In the example shown in FIG. 6, the supported portion detecting function 1243 detects the left knee P1 of the subject P as the supported portion. The supported portion detecting function 1243 causes the memory 121 to store supported portion detection data indicating the detected supported portion. For example, the supported portion detection data is data indicating the location (i.e., the range of coordinates) of the detected supported portion on the air mattress 111.

If the subject P is not detected (step S5: No), the subject detecting function 1242 repeats imaging of the bed 105 using the ceiling camera 115 (step S3).

After the supported portion is detected, the air-supply-target-air-cell determining function 1244 determines air cells to be supplied with air corresponding to the detected supported portion (step S7). Specifically, the air-supply-target-air-cell determining function 1244 determines the air cells to be supplied with air based on the image taken by the ceiling camera 115 and the supported portion determined by the supported portion determining function 1241. More specifically, the air-supply-target-air-cell determining function 1244 determines the air cells to be supplied with air based on the supported portion detected from the subject P in the taken image detected by the supported portion detecting function 1243. More specifically, the air-supply-target-air-cell determining function 1244 reads from the memory 121 the supported portion detection data and position information of the air cells 1111 that has been stored in advance. The air-supply-target-air-cell determining function 1244 then compares the read supported portion detection data with the position information of the air cells 1111 to select air cells 1111 that at least match in position the supported portion as the air cells to be supplied with air.

If only air cells 1111 that match in position the supported portion are to be inflated, a gap may be formed between a boundary of the supported portion and the top surface 1110 of the air mattress 111. If the gap is big, the subject P may be supported in an uncomfortable position, and it may make it difficult for the reception coil 111b to appropriately receive the magnetic resonance signal. Therefore, the air-supply-target-air-cell determining function 1244 may also determine a predetermined number of air cells 1111 located around the supported portion as the air cells to be supplied with air, in addition to the air cells 1111 corresponding to the supported portion. In the example shown in FIG. 6, the air-supply-target-air-cell determining function 1244 selects the air cells 1111 corresponding to the left knee P1 and a predetermined number of air cells 1111 around the left knee P1 as the air cells to be supplied with air 1111A.

It may be possible to prevent a big gap from being formed between the periphery of the supported portion and the top surface 1110 of the air mattress 111 by including the air cells 1111 around the supported portion in the air cells to be supplied with air. As a result, the inconvenience of the subject P may be alleviated, and the reception coil 111b may appropriately receive the magnetic resonance signal to improve the quality of the medical image. The memory 121 may store data indicating the correspondence relationship between the types of the supported portion and the range (numbers) of air cells 1111 to which air is supplied. In this case, the air-supply-target-air-cell determining function 1244 may easily determine the range of air cells 1111 around the supported portion, to which air is supplied, based on the data indicating the correspondence relationship stored in the memory 121.

The air-supply-target-air-cell determining function 1244 causes the memory 121 to store air cells to be supplied with air data indicating the determined air cells to be supplied with air.

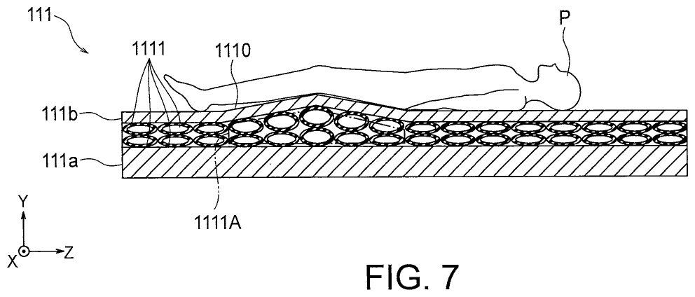

FIG. 7 is an explanatory diagram for explaining a step of supplying air to the air cells to be supplied with air in the example of the operation of the magnetic resonance imaging apparatus 10 according to the first embodiment. After the air cells to be supplied with air are determined, the air supply control function 1245 supplies air to the determined air cells to be supplied with air (step S8). Specifically, the air supply control function 1245 reads the air cells to be supplied with air data from the memory 121. Based on the read air cells to be supplied with air data, the air supply control function 1245 outputs a control signal instructing the valve control circuitry 114 to open the valves 1112 connected to the air cells to be supplied with air, and a control signal instructing the start of air supply to the air pump control circuitry 113. As a result, the air supply control function 1245 starts supplying air to the air cells to be supplied with air using the valves 1112 and the air pump 112. When air is supplied for a period of time that is needed for supplying a predetermined amount of air to the air cells to be supplied with air, the air supply control function 1245 outputs a control signal to the valve control circuitry 114 instructing to close the valves 1112 connected to the air cells to be supplied with air, and a control signal instructing the end of air supply to the air pump control circuitry 113. In this manner, the air supply control function 1245 stops the air supply to the air cells to be supplied with air using the valves 1112 and the air pump 112.

The memory 121 may store in advance data indicating the correspondence relationship between the type of supported portion, the range (number) of air cells 1111 to which air is supplied, and the amount of air supplied to each of the air cells 1111. In this case, the air supply control function 1245 may easily determine the amount of air supplied to the air cells to be supplied with air based on the data indicating the correspondence relationship read from the memory 121. When air is supplied to the air cells to be supplied with air, the air cells to be supplied with air may inflate, which may cause a portion of the top surface 1110 of the air mattress 111 corresponding to the air cells to be supplied with air 1111A to protrude upward as shown in FIG. 7. As a result, the supported portion of the subject P may be appropriately supported. As shown in FIG. 7, the amount of air supplied to an upper layer side of the air cells 1111 may be greater than the amount of air supplied to a lower layer side of the air cells 1111 in the region of the air cells to be supplied with air. Alternatively, the amount of air supplied to the lower layer side of the air cells 1111 may be greater than the amount of air supplied to the upper layer side of the air cells 1111.

After air is supplied to the air cells to be supplied with air, the top panel driver 1050 moves the top panel 105a together with the subject P on the air mattress 111 to the imaging space of the gantry 100 under the control of the bed control circuitry 109. In the imaging space, an image of the subject P is magnetically taken. After the image is taken, the air exhaustion control function 1246 exhausts air from the air cells to be supplied with air (step S9). Specifically, the air supply control function 1245 outputs a control signal to the valve control circuitry 114 to instruct the valve control circuitry 114 to open the valves 1112 connected to the air cells to be supplied with air and to open the exhaust valve 1114, thereby exhausting air from the air cells to be supplied with air.

As described above, in the first embodiment, the air supply control function 1245 controls the supply of air to the air cells 1111 of the air mattress 111 so as to partially protrude the top surface 1110 of the air mattress 111 upward from the flat state.

Thus, it is not necessary for the operator to put a mattress between the bed 105 and the subject P. Therefore, it is possible to reduce the load of the operator and shorten the time needed for the preparation for the imaging.

Furthermore, in the first embodiment, the air-supply-target-air-cell determining function 1244 determines the air cells to be supplied with air based on the image taken by the ceiling camera 115. Thereafter, the air supply control function 1245 supplies air to the determined air cells to be supplied with air.

Thus, it is possible to determine the air cells to be supplied with air easily, and to further shorten the time needed for the preparation of the imaging.

Furthermore, in the first embodiment, the air-supply-target-air-cell determining function 1244 determines the air cells to be supplied with air based on the image taken by the ceiling camera 115 and the supported portion determined by the supported portion determining function 1241.

Thus, the air cells to be supplied with air may be appropriately determined based on the supported portion.

Furthermore, in the first embodiment, the supported portion determining function 1241 determines the supported portion using the order information.

Thus, the supported portion may be determined easily and appropriately.

In the first embodiment, the subject detecting function 1242 detects the subject P on the air mattress 111 based on the image taken by the ceiling camera 115. Furthermore, the supported portion detecting function 1243 detects the supported portion determined by the supported portion determining function 1241 in the subject P detected by the subject detecting function 1242. The air-supply-target-air-cell determining function 1244 determines the air cells to be supplied with air based on the supported portion detected by the supported portion detecting function 1243.

Therefore, the air cells to be supplied with air may be further appropriately determined.

### Second Embodiment

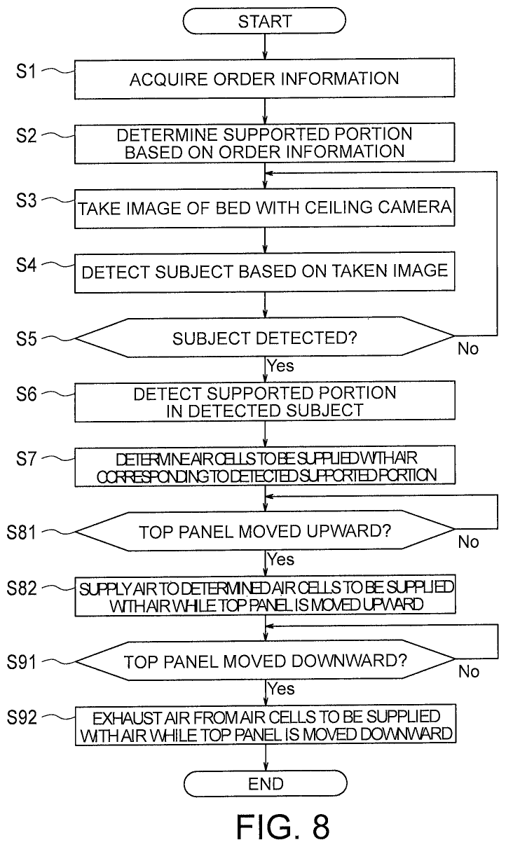

Next, a second embodiment will be described, in which the supply and the exhaustion of air to and from the air cells 1111 are controlled as the top panel 105a vertically moves. FIG. 8 is a flowchart showing an example of an operation of a magnetic resonance imaging apparatus 10 according to the second embodiment.

As shown in FIG. 8, in the second embodiment, the air supply control function 1245 determines whether the top panel 105a is to be lifted by the top panel driver 1050 to take an image of the subject P (step S81) after the air cells to be supplied with air are determined (step S7). For example, the air supply control function 1245 determines whether the top panel 105a is to be lifted based on whether an electrical signal instructing the lifting of the top panel 105a is outputted from the input interface 123 to the processing circuitry 124.

If it is determined that the top panel 105a is to be lifted (step S81: Yes), the air supply control function 1245 supplies air to the air cells to be supplied with air while the top panel 105a is being lifted (step S82). Specifically, the air supply control function 1245 reads the air cells to be supplied with air data from the memory 121, and outputs a control signal to the valve control circuitry 114 instructing the opening of the valves 1112 connected to the corresponding air cells to be supplied with air based on the read air cells to be supplied with air data, and also outputs a control signal to the air pump control circuitry 113 instructing the start of air supply. In this manner, the air supply control function 1245 starts supplying air to the air cells to be supplied with air using the valves 1112 and the air pump 112.

At this time, the air supply control function 1245 may control the speed in supplying air (i.e., the amount of air supplied in a unit time) to the air cells to be supplied with air so that the supply of air to the air cells to be supplied with air may be finished while the top panel 105a is being lifted. The speed in supplying air may be a speed at which the supply of air to the air cells to be supplied with air may be finished at or before the end of the period of time during which the top panel 105a is lifted. If the speed in supplying air is controlled, the air supply control function 1245 may calculate the speed in supplying air based on the time needed for lifting the top panel 105a, which is stored in the memory 121 in advance, and a target amount of air supply to the air cells to be supplied with air, which is also stored in the memory 121 in advance. Since the target amount of air supply may differ in each of the air cells 1111 included in the air cells to be supplied with air, the speed in supplying air may also differ for each of the air cells 1111. The air supply control function 1245 may control the power supply to the air pump 112 via the air pump control circuitry 113 or the degree of opening of the valves 1112 via the valve control circuitry 114 in accordance with the calculated speed in supplying air.

When the time at which the lifting of the top panel 105a may be finished is reached, the air supply control function 1245 outputs a control signal to the valve control circuitry 114 instructing the closing of the valves 1112 connected to the air cells to be supplied with air and, and also outputs a control signal to the air pump control circuitry 113 instructing the end of air supply. In this manner, the air supply control function 1245 finishes the supply of air to the air cells to be supplied with air using the valves 1112 and the air pump 112.

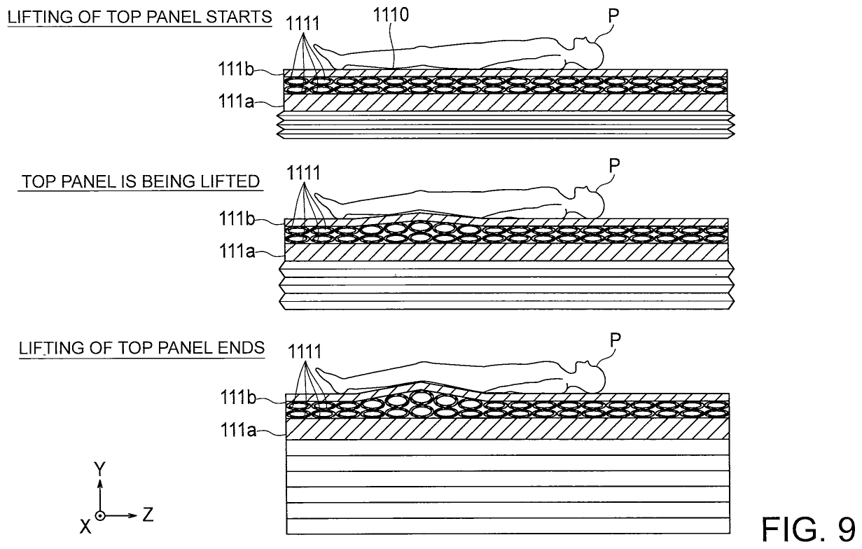

FIG. 9 is an explanatory diagram for explaining a step of supplying air to the air cells to be supplied with air in an example of an operation of the magnetic resonance imaging apparatus 10 according to the second embodiment. The above-described operation performed by the air supply control function 1245 enables the air cells to be supplied with air to start inflating when the top panel 105a starts being lifted, thereby causing the top surface 1110 of the air mattress 111 to protrude at a portion corresponding to the air cells to be supplied with air, for example, as shown in FIG. 9. After the lifting of the top panel 105a is finished, the inflating of the air cells to be supplied with air may be ended so that the portion of the top surface 1110 of the air mattress 111 corresponding to the air cells to be supplied with air may stop protruding.

If it is determined that the top panel 105a is not to be lifted (step S81: No), the air supply control function 1245 repeats the determination of whether the top panel 105a is to be lifted (step S81).

After an image of the subject P is taken in the imaging space, the air exhaustion control function 1246 determines whether the top panel 105a is to be lowered by the top panel driver 1050 (step S91). For example, the air supply control function 1245 determines whether the top panel 105a is to be lowered based on whether an electrical signal is inputted from the input interface 123 instructing the lowering of the top panel 105a.

If it is determined that the top panel 105a is to be lowered (step S91: Yes), the air exhaustion control function 1246 exhausts air from the air cells to be supplied with air while the top panel 105a is being lowered (step S92). Specifically, the air exhaustion control function 1246 outputs a control signal instructing the opening of the valves 1112 connected to the air cells to be supplied with air and a control signal instructing the opening of the exhaust valve 1114 to the valve control circuitry 114 in order to exhaust air from the air cells to be supplied with air.

At this time, the air exhaustion control function 1246 may control the speed in exhausting air (i.e., the amount of air exhausted in a unit time) from the air cells to be supplied with air so that the exhaustion of air from the air cells to be supplied with air may be finished while the top panel 105a is being lowered. In this case, the air exhaustion control function 1246 may calculate the speed in exhausting air based on the time needed for lowering the top panel 105a, which is stored in the memory 121 in advance, and a target amount of air to be supplied to the air cells to be supplied with air, which is also stored in the memory 121 in advance. Since the target amount of air supply may differ in each of the air cells 1111 included in the air cells to be supplied with air, the speed in exhausting air may also differ for each of the air cells 1111. The air supply control function 1245 may control the degree of opening of the valves 1112 and the exhaust valve 1114 via the valve control circuitry 114 in accordance with the calculated speed in exhausting air.

If it is determined that the top panel 105a is not to be lowered (step S91: No), the air exhaustion control function 1246 repeatedly determines whether the top panel 105a is to be lowered (step S91).

As described above, in the second embodiment, the air supply control function 1245 supplies air to the air cells to be supplied with air determined by the air-supply-target-air-cell determining function 1244 while the top panel 105a is lifted by the top panel driver 1050. The air exhaustion control function 1246 exhausts air from the air cells to be supplied with air while the top panel 105a is lowered by the top panel driver 1050.

As a result, air may be supplied to the air cells to be supplied with air in parallel to the lifting of the top panel 105a. Therefore, it is possible to further shorten the time needed for the preparation of the imaging. Furthermore, since air may be exhausted from the air cells to be supplied with air in parallel to the lowering of the top panel 105a, it is possible to shorten the time needed for the work after the imaging is finished.

### Third Embodiment

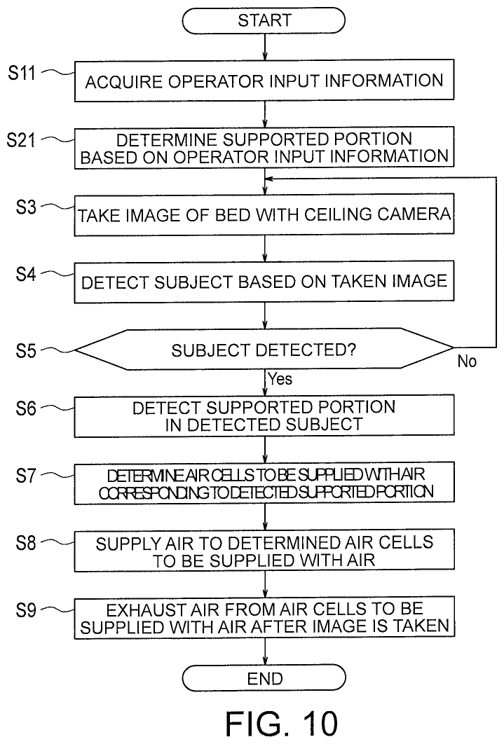

A third embodiment will be described below, in which a supported portion is determined based on input information inputted by the operator. FIG. 10 is a flowchart showing an example of an operation of a magnetic resonance imaging apparatus 10 according to the third embodiment. In the first embodiment, the supported portion determining function 1241 determines a supported portion based on order information. In contrast, in the third embodiment, the supported portion determining function 1241 determines a supported portion based on operator input information relating to the supported portion, inputted by the operator.

Specifically, as shown in FIG. 10, the supported portion determining function 1241 acquires operator input information inputted to the processing circuitry 124 (step S11). The operator input information designates a supported portion such as “knee.” The operator input information may be inputted via the input interface 123, or via a GUI shown on the display 122.

After acquiring the operator input information, the supported portion determining function 1241 determines a supported portion based on the acquired operator input information (step S21). The supported portion determining function 1241 causes the memory 121 to store supported portion data indicating the determined supported portion. At and after step S3, the operation of the third embodiment is the same as the first embodiment.

As described above, in the third embodiment, the supported portion determining function 1241 determines a supported portion based on the operator input information.

This improves the degree of freedom in selecting a supported portion.

### Fourth Embodiment

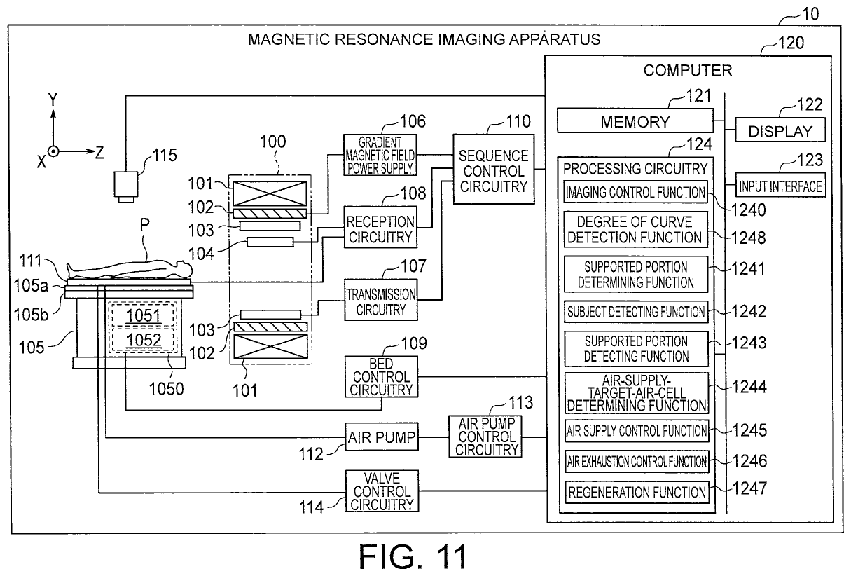

Next, a fourth embodiment will be described, in which air cells to be supplied with air are determined based on the degree of curve on the back side of a subject P. FIG. 11 is a block diagram showing an example of a configuration of the magnetic resonance imaging apparatus 10 according to the fourth embodiment.

As shown in FIG. 11, the processing circuitry 124 of the magnetic resonance imaging apparatus 10 according to the fourth embodiment includes a degree of curve detection function 1248 in addition to the functions of the processing circuitry 124 shown in FIG. 1. The degree of curve detection function 1248 detects the degree of curve on the back side of the subject P based on an image of the side view of the subject P taken by the ceiling camera 115. The ceiling camera 115 is an example of a second imaging device. The image of the side view of the subject P (“side view image”) is an example of a second image. The degree of curve on the back side of the subject P at least includes the degree of curve of the back of the subject P. The degree of curve on the back side of the subject P may include the degree of curve of a portion of the subject P other than the back. The degree of curve on the back side of the subject P may be detected based on an image taken by a camera other than the ceiling camera 115.

The air-supply-target-air-cell determining function 1244 determines the air cells to be supplied with air based on an image of a front side of the subject P taken by the ceiling camera 115 and the degree of curve on the back side of the subject P detected by the degree of curve detection function 1248. As shown in FIG. 6, the image of the front side of the subject P is an image of the subject P lying on the back on the air mattress 111, taken by the ceiling camera 115. In the fourth embodiment, this image is called “image of the front side” in order to avoid confusion with the image from the side view image of the subject P.

When air cells to be supplied with air are determined, first the supported portion determining function 1241 determines a supported portion based on the degree of curve on the back side of the subject P detected by the degree of curve detection function 1248, and then the air-supply-target-air-cell determining function 1244 determines air cells to be supplied with air based on the supported portion determined by the supported portion determining function 1241 and the image of the front side of the subject P taken by the ceiling camera 115. More specifically, the air-supply-target-air-cell determining function 1244 determines air cells to be supplied with air based on the image of the supported portion detected by the supported portion detecting function 1243 in the image of the front side of the subject P.

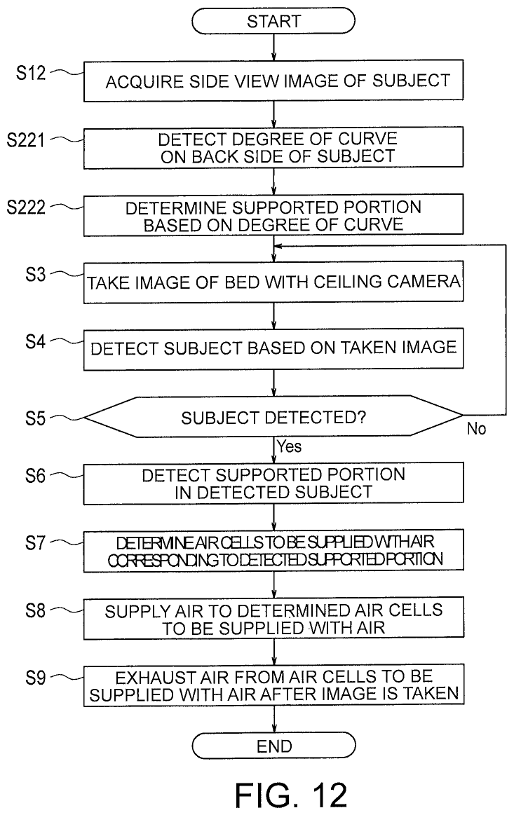

Next, an example of an operation of the magnetic resonance imaging apparatus 10 according to the fourth embodiment having the above-described configuration will be described. FIG. 12 is a flowchart showing an example of an operation of the magnetic resonance imaging apparatus 10 according to the fourth embodiment.

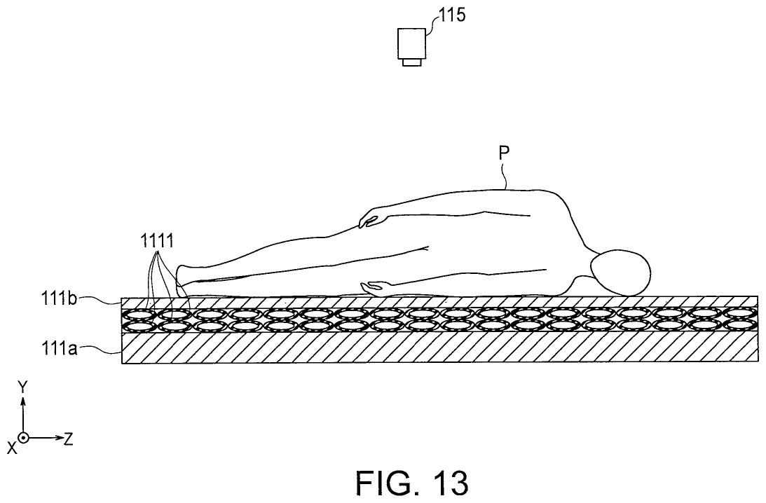

As shown in FIG. 12, first, the degree of curve detection function 1248 acquires a side view image of the subject P from the ceiling camera 115 (step S12). FIG. 13 is an explanatory diagram for explaining a step of acquiring a side view image of the subject P in the example of the operation of the magnetic resonance imaging apparatus 10 according to the fourth embodiment. As shown in FIG. 13, the side view image of the subject P is taken when the subject P lies on one side on the air mattress 111 and the ceiling camera 115 takes an image of the subject P from above. The side view image of the subject P may be obtained by taking an image of subject P lying on the back on the air mattress 111 from a side by a camera that is different from the ceiling camera 115. The air supply control function 1245 causes the memory 121 to store the side view image of the subject P taken by the ceiling camera 115.

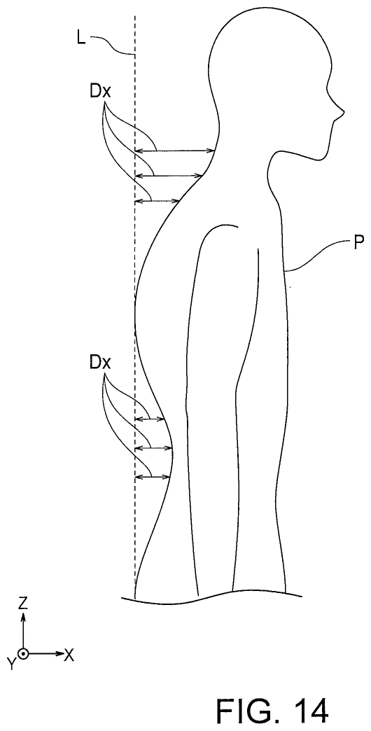

After the side view image of the subject P is taken, the degree of curve detection function 1248 detects the degree of curve on the back side of the subject P based on the side view image of the subject P (step S221). FIG. 14 is an explanatory diagram for explaining a step of detecting the degree of curve on the back side of the subject P in the magnetic resonance imaging apparatus 10 according to the fourth embodiment. For example, the degree of curve detection function 1248 defines a reference line L that extends in the Z axis direction, passing through a furthest point on the back side (in the negative direction on the X axis) in the side view image of the subject P read from the memory 121, as shown in FIG. 14. After defining the reference line L, the degree of curve detection function 1248 detects a distance Dx (in the X axis direction) from the reference line L to each portion of the back side as the degree of curve. The way of defining the degree of curve is not limited to the manner described above. For example, the degree of curve detection function 1248 may detect the amount of change in the distance Dx in the Z axis direction. The degree of curve detection function 1248 causes the memory 121 to store degree of curve data indicating the degree of curve on the back side of the subject P.

After the degree of curve on the back side of the subject P is detected, the supported portion determining function 1241 determines a supported portion based on the detected degree of curve, as shown in FIG. 12 (step S222). Specifically, the supported portion determining function 1241 reads the degree of curve data from the memory 121, and determines the supported portion based on the read degree of curve data. For example, the supported portion determining function 1241 may determine a portion of the subject P where the distance Dx from the reference line L to each point on the back side is equal to or greater than a threshold value as the supported portion. The threshold value may be 0 [cm].

The operation at and after step S3 is the same as that of the first embodiment. As in the first embodiment, the image of the front side of the subject P is taken at step S3.

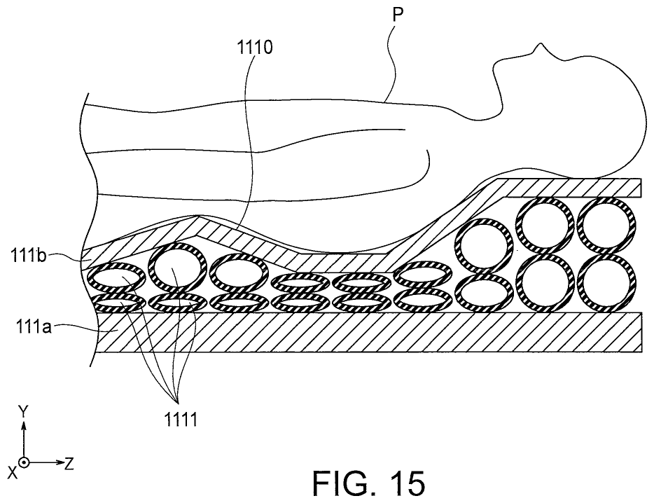

FIG. 15 is an explanatory diagram for explaining a step of supplying air to the air cells to be supplied with air in the example of the operation of the magnetic resonance imaging apparatus 10 according to the fourth embodiment. As described above, by determining the supported portion based on the degree of curve on the back side of the subject P, it is possible to determine air cells to be supplied with air suitable for the degree of curve on the back side. The memory 121 may store in advance data indicating the corresponding relationship between the degree of curve on the back side of the subject P and the amount of air supplied to the air cells 1111. This enables the air supply control function 1245 to easily determine the amount of air to be supplied to the air cells to be supplied with air in consideration of the degree of curve on the back side based on the data indicating the corresponding relationship stored in the memory 121. By determining the air cells to be supplied with air based on the degree of curve on the back side of the subject P, it may be possible to protrude the corresponding portion of the top surface 1110 of the air mattress 111 substantially without a space between the top surface 1110 of the air mattress 111 and the back side of the subject P, as shown in FIG. 15.

As described above, in the fourth embodiment, the degree of curve detection function 1248 detects the degree of curve on the back side of the subject P based on the side view image of the subject P taken by the ceiling camera 115. The air-supply-target-air-cell determining function 1244 then determines the air cells to be supplied with air based on the image of the front side of the subject P taken by the ceiling camera 115 and the degree of curve on the back side of the subject P detected by the degree of curve detection function 1248.

As a result, it is possible to support the subject P in a convenient position in accordance with the degree of curve on the back side of the subject P, and to take an appropriate image of the subject P.

### Fifth Embodiment

A fifth embodiment will then be described below, in which air cells to be supplied with air and the amount of air supplied to the air cells to be supplied with air are determined based on subject-specific information.

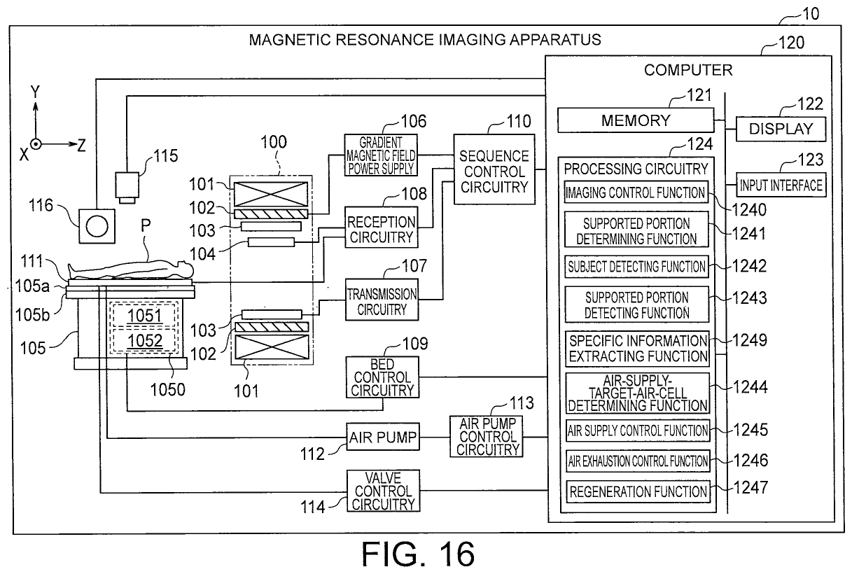

FIG. 16 is a block diagram showing an example of a configuration of a magnetic resonance imaging apparatus according to the fifth embodiment 10. As shown in FIG. 16, the magnetic resonance imaging apparatus 10 according to the fifth embodiment includes a side view camera 116 in addition to the configuration of the first embodiment. The side view camera 116 is an example of a third imaging device. Furthermore, the processing circuitry 124 of the magnetic resonance imaging apparatus 10 according to the fifth embodiment further includes specific information extracting function 1249 in addition to the functions of the processing circuitry 124 shown in FIG. 1.

The side view camera 116 is located so that a side of the subject P on the air cells 1111 is included in the viewing angle of the side view camera 116. For example, the side view camera 116 may be a camera including a solid-state imager such as a CMOS sensor. The side view camera 116 takes a side view image of the subject P on the air mattress 111 under the control of the computer system 120. The side view image of the subject P is an example of a third image. If an image of the subject P may be taken in an aspect of extracting subject-specific information, which will be described later, the image of the subject P may be taken by a camera other than the side view camera 116. For example, a side view image of the subject P may be taken by a camera disposed diagonally upward relative to the subject P, and coordinate transformation (view transformation) may be performed based on, for example, a camera parameter, thereby forming an image equivalent to the side view image. Alternatively, an infrared camera may be used to take a side view image of the subject P.

The specific information extracting function 1249 extracts subject-specific information indicating an external appearance characteristic specific to the subject P from the side view image of the subject P taken by the side view camera 116. For example, the subject-specific information may be body thickness information in the vertical direction of the subject P on the air mattress 111. Furthermore, for example, the specific information extracting function 1249 may extract bone structure information of the subject P on the air mattress 111 as the subject-specific information. The specific information extracting function 1249 may acquire a part of the subject-specific information from information other than the side view image of the subject P (.g., information inputted via the input interface 123 or the GUI on the display 122).

The air-supply-target-air-cell determining function 1244 determines air cells to be supplied with air and the amount of air supplied to the air cells to be supplied with air based on the image taken by the ceiling camera 115 and the subject-specific information extracted by the specific information extracting function 1249. Specifically, the air-supply-target-air-cell determining function 1244 determines the air cells to be supplied with air and the amount of air supplied to the air cells to be supplied with air based on the supported portion detected from the image by the supported portion detecting function 1243 and the subject-specific information.

The air supply control function 1245 supplies air to the air cells to be supplied with air based on the air cells to be supplied with air and the amount of air supplied to the air cells to be supplied with air determined by the air-supply-target-air-cell determining function 1244.

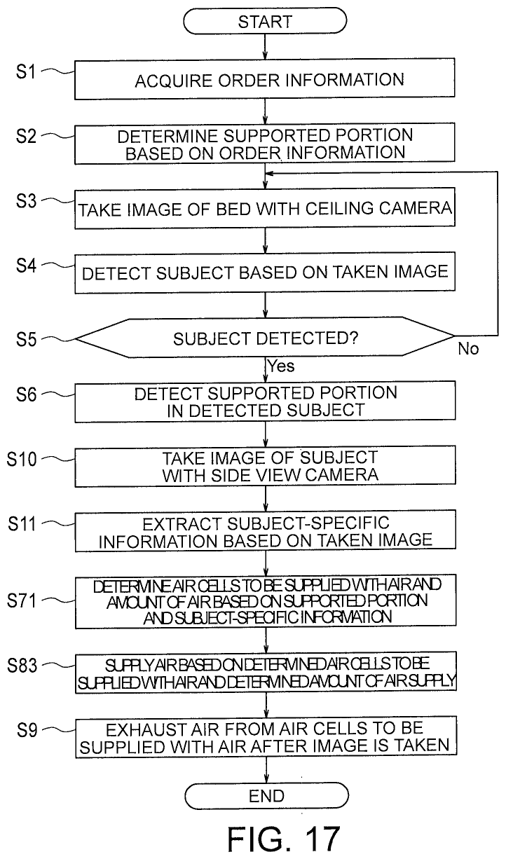

Next, an example of an operation of the magnetic resonance imaging apparatus 10 according to the fifth embodiment will be described. FIG. 17 is a flowchart showing the example of the operation of the magnetic resonance imaging apparatus 10 according to the fifth embodiment.

As shown in FIG. 17, in the fifth embodiment, the air supply control function 1245 causes the side view camera 116 to take the image of the subject P (step S10) after the supported portion detecting function 1243 detects the supported portion (step S6). The air supply control function 1245 causes the memory 121 to store the side view image of the subject P taken by the side view camera 116.

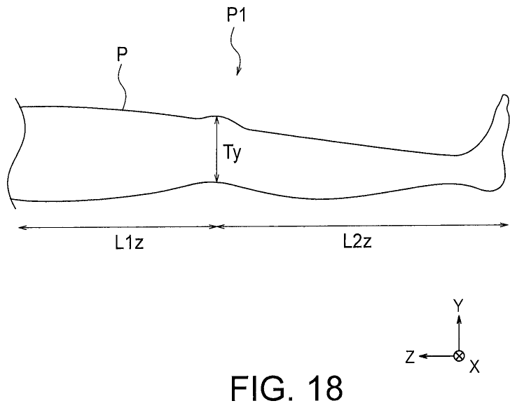

After the side view image of the subject P is taken, the specific information extracting function 1249 extracts the subject-specific information (step S11). Specifically, specific information extracting function 1249 reads the side view image of the subject P stored in the memory 121, and extracts the subject-specific information based on the read side view image. FIG. 18 is an explanatory diagram for explaining a step of extracting the subject-specific information in the example of the operation of the magnetic resonance imaging apparatus 10 according to the fifth embodiment. For example, as shown in FIG. 18, the specific information extracting function 1249 extracts body thickness information Ty in the vertical direction (Y axis direction) of the subject P on the air mattress 111 and the bone structure information (L1z, L2z) as the subject-specific information. In the example shown in FIG. 18, the body thickness information Ty corresponds to the thickness of the knee, which is the supported portion P1 and also the imaged portion of the subject P, in the Y axis direction. The bone structure information (L1z, L2z) includes a length of a femoral area (i.e., “over-the-knee area”) in the Z axis direction of the subject P, and a length L2z of a leg area (i.e., “under-the-knee area”) in the Z axis direction of the subject P. The specific information extracting function 1249 causes the memory 121 to store the extracted subject-specific information.

After the subject-specific information is extracted, as shown in FIG. 17, the air-supply-target-air-cell determining function 1244 determines the air cells to be supplied with air and the amount of air supplied to the air cells to be supplied with air (step S71). Specifically, the air-supply-target-air-cell determining function 1244 reads the supported portion detection data (i.e., the detected supported portion) stored in the memory 121 at step S6 and the subject-specific information stored in the memory 121 at step S11, and determines the air cells to be supplied with air and the amount of air based on the supported portion data and the subject-specific information.

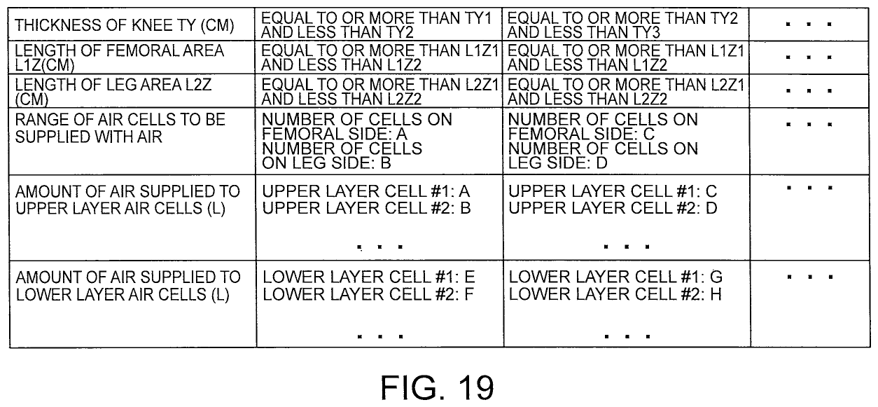

FIG. 19 is an explanatory diagram for explaining a step of determining the air cells to be supplied with air and the amount of air in the example of the operation of the magnetic resonance imaging apparatus 10 according to the fifth embodiment. For example, as shown in FIG. 19, the air-supply-target-air-cell determining function 1244 may determine the amount of air supplied to the air cells to be supplied with air based on data indicating the corresponding relationship between the subject-specific information, the area of the air cells to be supplied with air, and the amount of air to be supplied. FIG. 19 shows the correspondence relationship among the range of the thickness Ty of the knee, the range of the length L1z of the femoral area, the range of the length L2z of the leg area, the range of the air cells to be supplied with air, the amount of air supplied to the upper layer air cells, and the amount of air supplied to the lower layer air cells. In FIG. 19, “Upper Layer Air Cells” means the air cells 1111 in the upper layer of the vertically arranged two layers shown in FIG. 3. In FIG. 19, “Lower Layer Air Cells” means the air cells 1111 in the lower layer of the vertically arranged two layers shown in FIG. 3. Furthermore, in FIG. 19, “Number of Cells on Femoral Side” and “Number of Cells on Leg Side in “Range of Air Cells to be Supplied with Air” indicate the total number of upper layer air cells and lower layer air cells located in the femoral area or the leg area. FIG. 19 shows an example in which the range of the length L1z of the femoral area and the range of the length L2z of the leg area are fixed, and the range of the thickness Ty of the knee is changed. As a result, the amount of supplied air changes. The reason for the change in amount of supplied air is not limited to the above case, but the amount of supplied air may be changed when at least one of the range of the length L1z of the femoral area and the range of the length L2z of the leg area is changed.

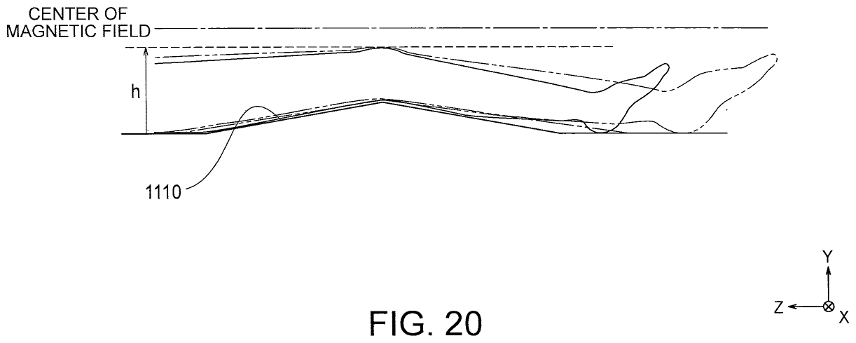

FIG. 20 is an explanatory diagram for explaining a step of determining an amount of air to be supplied in the example of the operation in the magnetic resonance imaging apparatus 10 according to the fifth embodiment. The amount of supplied air shown in FIG. 19 is intended to cause the top surface 1110 of the air mattress 111 to protrude so that substantially no space is formed between the supported portion and the top surface 1110 of the air mattress 111. The amount of supplied air shown in FIG. 19 may be intended to support an area above the knee to reach a predetermined height h that is close to the center of the magnetic field in the imaging space as shown in FIG. 20, even if the subject-specific information may differ for each subject P. In this case, the imaged portion may be located near the center of the magnetic field regardless of the physical difference of the subject P. Therefore, a medical image with good image quality may be obtained. If an X-ray CT apparatus is used as the medical image diagnosis apparatus, the amount of supplied air may be set in the same manner as shown in FIGS. 19 and 20. In this case, the amount of supplied air may be set to support the portion above the knee at a predetermined height close to a center of rotation of an X-ray tube and an X-ray detector in the gantry. As a result, a CT image with good image quality may be obtained.

The air-supply-target-air-cell determining function 1244 causes the memory 121 to store the air cells to be supplied with air data indicating the determined air cells to be supplied with air and air supply amount data indicating the determined amount of air supply.

After the air cells to be supplied with air and the amount of air supplied to the air cells to be supplied with air are determined, the air supply control function 1245 supplies air to the air cells to be supplied with air, as shown in FIG. 17 (step S83). Specifically, the air supply control function 1245 reads the air cells to be supplied with air data and the air supply amount data stored in the memory 121, and supplies air to the air cells to be supplied with air according to the read air cells to be supplied with air data and the read air supply amount data. For example, the air supply control function 1245 outputs a control signal to the valve control circuitry 114 instructing the opening of the valves 1112 connected to the air cells to be supplied with air indicated by the air cells to be supplied with air data, and a control signal to the air pump control circuitry 113 indicating the start of air supply. In this manner, the air supply control function 1245 starts supplying air to the air cells to be supplied with air using the valves 1112 and the air pump 112. When the end of the period of time needed to supply the amount of air shown in the air supply amount data is approaching, the air supply control function 1245 outputs a control signal to the valve control circuitry 114 instructing the closing of the valves 1112 connected to the air cells to be supplied with air, and a control signal to the air pump control circuitry 113 instructing the end of the air supply. In this manner, the air supply control function 1245 ends the supply of air to the air cells to be supplied with air using the valves 1112 and the air pump 112. Thereafter, step S9 is performed in the same manner as that in the first embodiment.

As described above, in the fifth embodiment, the specific information extracting function 1249 extracts the subject-specific information based on the side view image of the subject P taken by the side view camera 116. The air-supply-target-air-cell determining function 1244 determines the air cells to be supplied with air and the amount of air supplied to the air cells to be supplied with air based on the image of the subject P taken by the ceiling camera 115 and the subject-specific information extracted by the specific information extracting function 1249. The air supply control function 1245 supplies air to the air cells to be supplied with air based on the air cells to be supplied with air and the amount of air determined by the air-supply-target-air-cell determining function 1244.

Thus, it may be possible to support the subject P in a position that is not inconvenient to the subject P regardless of the physical difference of the subject P. It may also be possible to appropriately take an image of the subject P.

The term “processor” used in the above descriptions may mean circuitry such as a central processing unit (CPU), a graphics processing unit (GPU), or an application specific integrated circuit (ASIC), a programmable logic device like a simple programmable logic device (SPLD) or a complex programmable logic device (CPLD), or a field programmable gate array (FPGA). The processor performs a function by reading a program stored in storage circuity and executing the program. Instead of storing a program in the storage circuity, the processor may have the program in its circuitry. In this case, the processor performs the function by reading and performing the program set in the circuitry. The processor is not limited to a single circuit processor, but may be a combination of a plurality of independent circuits formed as a single processor to perform the function. The components shown in FIG. 1 may be implemented to a single processor to perform their functions.

According to one or more embodiments described above, it is possible to shorten the time needed for the preparation for the imaging while reducing the load of the operator.

While certain embodiments have been described, these embodiments have been presented by way of example only, and are not intended to limit the scope of the inventions. The novel apparatuses and methods described herein may be performed in a variety of other forms. Furthermore, omissions, substitutions, changes and combinations may be made in the embodiments of the apparatuses and methods described herein without departing from the spirit of the inventions. Such embodiments and modifications thereof are intended to be covered by the scope and spirit of the inventions as well as the claimed inventions and their equivalents.

What is claimed is:

1. A medical image diagnosis apparatus comprising:
an air mattress configured to be placed on a bed, the air mattress including a plurality of air cells configured to partition an inside of the air mattress into a plurality of regions to seal air supplied to the air cells;
an air supplier configured to supply air to the air cells of the air mattress; and
processing circuitry configured to control a supply of air to the air cells of the air mattress so that a top surface of the air mattress partially protrudes upward from a flat state of the air mattress.

2. The medical image diagnosis apparatus according to claim 1, further comprising an imaging device configured to image a subject on the air mattress, wherein
the processing circuitry is further configured to
determine air cells to be supplied with air among the air cells of the air mattress based on an image taken by the imaging device, and
supply air to the determined air cells.

3. The medical image diagnosis apparatus according to claim 2, wherein the processing circuitry is further configured to
determine a supported portion of the subject, wherein the supported portion is supported when the top surface of the air mattress protrudes upward based on input information, and
determine the air cells to be supplied with air based on the image taken by the imaging device and the determined supported portion.

4. The medical image diagnosis apparatus according to claim 3, wherein the processing circuitry is further configured to determine the supported portion using order information indicating an imaged portion of the subject in a gantry as the input information.

5. The medical image diagnosis apparatus according to claim 3, wherein the processing circuitry is further configured to determine the supported portion using information regarding the supported portion inputted by an operator as the input information.

6. The medical image diagnosis apparatus according to claim 2, further comprising a second imaging device configured to take an image of the subject on the air mattress, wherein
the processing circuitry is further configured to
detect a degree of curve on a back side of the subject based on a second image taken by the second imaging device, and
determine the air cells to which air is supplied based on the image taken by the imaging device and the detected degree of curve on the back side of the subject.

7. The medical image diagnosis apparatus according to claim 3, wherein the processing circuitry is further configured to
detect the subject on the air mattress based on the image taken by the imaging device,
detect the determined supported portion from detected subject, and
determine air cells to be supplied with air based on the detected supported portion.

8. The medical image diagnosis apparatus according to claim 2, further comprising:
the bed; and
a gantry including an imaging space used for taking an image of the subject,
wherein the bed includes a top panel on which the air mattress is placed, and a top panel driver configured to drive the top panel in a vertical direction and a horizontal direction of the top panel,
wherein when the image of the subject is taken, the top panel driver lifts the top panel to a lift position corresponding to a height of the imaging space of the gantry, and moves the top panel along the longitudinal direction of the top panel from the lift position to the imaging space of the gantry, and
wherein the processing circuitry is further configured to supply air to the determined air cells when the top panel is lifted by the top panel driver.

9. The medical image diagnosis apparatus according to claim 8, further comprising an air exhauster configured to cause the air cells of the air mattress to exhaust air, wherein
the processing circuitry is further configured to control exhaustion of air from the air cells,
after the image of the subject is taken, the top panel driver returns the top panel from the imaging space of the gantry to the lift position and lowers the top panel from the lift position, and
the processing circuitry is further configured to, when the top panel is lowered, cause the air cells, which have been supplied with air, to exhaust air.

10. The medical image diagnosis apparatus according to claim 2, further comprising a third imaging device configured to take an image of the subject on the air mattress, wherein
the processing circuitry is further configured to
extract subject-specific information indicating an external appearance characteristic specific to the subject taken by the third imaging device,
determine the air cells to be supplied with air and an amount of air supplied to the air cells to be supplied with air based on the image taken by the imaging device and the extracted subject-specific information, and
supply air to the determined air cells, based on the determined air cells and a determined amount of air.

11. The medical image diagnosis apparatus according to claim 10, wherein the processing circuitry is further configured to extract information on a body thickness in a vertical direction of the subject on the air mattress as the subject-specific information.

12. The medical image diagnosis apparatus according to claim 10, wherein the processing circuitry is further configured to extract bone structure information of the subject on the air mattress as the subject-specific information.

13. The medical image diagnosis apparatus according to claim 2, wherein the air cells are arranged at least in a horizontal direction.

14. The medical image diagnosis apparatus according to claim 13, wherein the air cells are arranged in the horizontal direction and a vertical direction.

15. The medical image diagnosis apparatus according to claim 8, wherein in a plan view, the air cells are axis-symmetrically arranged relative to a center line of the air mattress extending in a longitudinal direction of the top panel.

16. The medical image diagnosis apparatus according to claim 1, wherein the air mattress further includes a reception coil configured to receive a magnetic resonance signal sent from a subject when an image of the subject is taken.

17. The medical image diagnosis apparatus according to claim 3, wherein the supported portion is a knee, a back, or a neck of the subject.

18. The medical image diagnosis apparatus according to claim 3, wherein the processing circuitry is further configured to
determine an amount of air supplied to the determined air cells based on correspondence information, which is stored in a memory, indicating a corresponding relationship among a type of the supported portion, a number of air cells to which air is supplied, and an amount of air to be supplied to each of the air cells to be supplied with air, and
supply the determined amount of air to the determined air cells.

19. The medical image diagnosis apparatus according to claim 6, wherein the processing circuitry is further configured to
determine an amount of air supplied to the determined air cells based on second correspondence information, which is stored in a second memory, indicating a corresponding relationship between a degree of curve on a back side of the subject and the amount of air supplied to the air cells, and
supply the determined amount of air to the determined air cells.

20. The medical image diagnosis apparatus according to claim 7, wherein the processing circuitry is further configured to determine air cells that match in position of the detected supported portion and a predetermined number of air cells around the air cells that match in position of the determined supported portion as the air cells to be supplied with air.
